(19) United States
(12) Patent Application Publication
(10) Pub. No.: US 2008/0254065 A1
(43) Pub. Date: Oct. 16, 2008

(54) Influenza Virus Vaccines

(75) Inventors: Audino Podda, Siena (IT); Olga Popova, Siena (IT); Francesca Piccinetti, Siena (IT)

Correspondence Address: NOVARTIS VACCINES AND DIAGNOSTICS INC., INTELLECTUAL PROPERTY R338, P.O. BOX 8097, Emeryville, CA, 94662-8097, US

(73) Assignee: CHIRON CORPORATION, Emeryville, CA (US)

(21) Appl. No.: 10/592,092

(22) PCT Filed: Mar. 9, 2005
(86) PCT No.: PCT/US05/08005

§ 371 Date: Dec. 5, 2007

Related U.S. Application Data
(60) Provisional application No. 60/551,897, filed on Mar. 9, 2004.
(60) Provisional application No. 60/556,534, filed on Mar. 25, 2004.

(51) Int. Cl. A61K 39/145; A61P 37/00

(52) U.S. Cl. 424/206.1

(57) ABSTRACT

The invention provides a vaccine for protecting a human patient against infection by a human influenza virus strain, wherein the vaccine comprises an antigen from an avian influenza virus strain that can cause highly pathogenic avian influenza. The antigen can invoke an antibody response in the patient that is capable of neutralising said human influenza virus strain. Whereas the prior art used known non-pathogenic avian strains to generate antibodies in humans against known pathogenic avian strains, the invention uses known pathogenic avian strains to protect against emerging pathogenic human strains. Furthermore, whereas the prior art focused on achieving a close antigenic match between the vaccine strain and the target strain, the invention selects vaccine strains based on their pathogenicity, regardless of any perceived close antigenic relationship to the target strain. As the invention does not require detailed knowledge of an emerging strain, a vaccine can be provided further in advance to reduce the risk and potential effects of a human pandemic outbreak.

All documents cited herein are incorporated by reference in their entirety.

### TECHNICAL FIELD

This invention is in the field of vaccination against influenza virus, and in particular vaccination against pandemic strains of influenza virus.

### BACKGROUND ART

In 1997, 2003, and again in 2004, antigenically-distinct avian H5N1 influenza viruses emerged as pandemic threats to human beings. During each of these outbreaks there was concern that the avian viruses would adapt to become transmissible from human to human. The optimum way of dealing with a human pandemic virus would be to provide a clinically approved well-matched vaccine (i.e., containing the hemagglutinin and neuraminidase antigens of the human pandemic strain), but this cannot easily be achieved on an adequate timescale.

One way of providing an effective vaccine against a new pandemic strain is to use antigens from an existing strain that is antigenically closely related to the new strain. For example, Nicholson et al. (The Lancet (2001) 357:1937-1943) described the use of antigens from the non-pathogenic A/Duck/Singapore/97 (H5N3) avian strain for vaccinating against the antigenically-related but pathogenic A/Hong Kong/156/97 (H5N1) strain. The authors were able to achieve neutralising antibody levels in immunised humans against the pathogenic avian strain.

This prior art approach to selecting strains for immunisation relies on knowing characteristics of a new strain, such as its antigenic profile, as this knowledge is required in order to select a suitable vaccine strain from strains that are already known. It is an object of the invention to provide further and improved ways of providing vaccines against emerging future human pandemic influenza virus strains, and in particular to provide ways that do not require detailed knowledge of antigenic characteristics of strains as they emerge as human pathogens.

### DISCLOSURE OF THE INVENTION

Whereas the prior art used known non-pathogenic avian strains to generate antibodies in humans against known pathogenic avian strains, the invention uses known pathogenic avian strains to protect against emerging pathogenic human strains. Furthermore, whereas the prior art focused on achieving a close antigenic match between the vaccine strain and the target strain, the invention selects vaccine strains based on their pathogenicity, regardless of any perceived close antigenic relationship to the target strain. As the invention does not require detailed knowledge of the antigenic profile of an emerging strain, a vaccine can be provided further in advance to reduce the risk and potential effects of a human pandemic outbreak.

Thus the invention provides a vaccine for protecting a human patient against infection by a human influenza virus strain, wherein the vaccine comprises an antigen from an avian influenza virus strain that can cause highly pathogenic avian influenza. The antigen can invoke an antibody response in the patient that is capable of neutralising not only the homologous vaccine strain, but also emerging heterologous human influenza vaccine strains. Preferably, the emerging heterologous human influenza vaccine will be within the same hemagglutinin type (i.e., H5 or H9) as the pathogenic avian influenza strain.

The invention also provides a process for preparing a vaccine for protecting a human patient against infection by a human influenza virus strain, comprising the step of admixing an antigen from an avian influenza virus strain that can cause highly pathogenic avian influenza with a pharmaceutically acceptable carrier and, optionally, with an adjuvant. Administration of the vaccine to the patient invokes an antibody response that is capable of neutralising said human influenza virus strain.

The invention also provides the use of an antigen from an avian influenza virus strain that can cause highly pathogenic avian influenza, in the manufacture of a vaccine for protecting a human patient against infection by a human influenza virus strain. The antigen in the vaccine can invoke an antibody response in the patient that is capable of neutralising said human influenza virus strain.

The invention also provides a method for protecting a human patient against infection by a human influenza virus strain, comprising the step of administering to the patient a vaccine that comprises an antigen from an avian influenza virus strain that can cause highly pathogenic avian influenza.

The invention also provides a vaccine comprising (a) an antigen from a pathogenic avian influenza virus strain, and optionally (b) antigen(s) from one or more (e.g. 1, 2 or 3) human influenza interpandemic virus strain(s). Component (b) in this vaccine may be a typical annual human influenza vaccine i.e. the invention provides a typical annual human influenza vaccine that is supplemented with an antigen from a pathogenic avian influenza virus strain. The vaccine may also include an adjuvant.

The invention also provides a process for preparing a vaccine, comprising the step of admixing (a) an antigen from a pathogenic avian influenza virus strain with (b) antigen(s) from one or more (e.g. 1, 2 or 3) human influenza virus strain(s). Component (a) will generally include an adjuvant; component (b) may or may not include an adjuvant. Similarly, the invention provides a kit comprising (a) a first container comprising an antigen from a pathogenic avian influenza virus strain with (b) a second container comprising antigen(s) from one or more (e.g. 1, 2 or 3) human influenza virus strain(s). Component (a) will generally include an adjuvant; component (b) may or may not include an adjuvant.

Avian antigens included in vaccines of the invention will generally be adjuvanted. As described below, two preferred adjuvants are (a) aluminium salts and (b) MF59.

### The Human Influenza Virus Strain

Vaccines of the invention use an avian antigen to protect patients against infection by an influenza virus strain that is capable of human-to-human transmission i.e. a strain that will spread geometrically or exponentially within a given human population without necessarily requiring physical contact. The patient may also be protected against strains that infect and cause disease in humans, but that are caught from birds rather than from other humans.

The invention is particularly useful for protecting against infection by pandemic, emerging pandemic and future pandemic human strains e.g. for protecting against H5 influenza subtypes. Depending on the particular season and on the nature of the antigen included in the vaccine, however, the invention may protect against other hemagglutinin subtypes, including H1, H2, H3, H4, H6, H7, H8, H9, H10, H11, H12, H13, H14, H15 or H16.

The characteristics of an influenza strain that give it the potential to cause a pandemic outbreak are: (a) it contains a new hemagglutinin compared to the hemagglutinins in currently-circulating human strains, i.e. one that has not been evident in the human population for over a decade (e.g. H2), or has not previously been seen at all in the human population (e.g. H5, H6 or H9, that have generally been found only in bird populations), such that the human population will be immunologically naive to the strain's hemagglutinin; (b) it is capable of being transmitted horizontally in the human population; and (c) it is pathogenic to humans.

As the invention protects against a strain that is capable of human-to-human transmission, the strain's genome will generally include at least one RNA segment that originated in a mammalian (e.g. in a human) influenza virus. Viruses in which all segments originated from avian viruses tend not to be capable of human-to-human transmission.

### The Avian Influenza Virus Strain

Vaccines of the invention include an antigen from an avian influenza virus strain. This strain is typically one that is capable of causing highly pathogenic avian influenza (HPAI). HPAI is a well-defined condition (Alexander Avian Dis (2003) 47(3 Suppl):976-81) that is characterized by sudden onset, severe illness and rapid death of affected birds/flocks, with a mortality rate that can approach 100%. Low pathogenicity (LPAI) and high pathogenicity strains are easily distinguished e.g. van der Goot et al. (Epidemiol Infect (2003) 131(2):1003-13) presented a comparative study of the transmission characteristics of low and high pathogenicity H5N2 avian strains.

For the 2004 season, examples of HPAI strains are H5N1 influenza A viruses e.g. A/Viet Nam/1196/04 strain (also known as A/Vietnam/3028/2004 or A/Vietnam/3028/04). Prior to 2004, the WHO lists HPAI strains as follows:

| | | Domestic birds | |
|---|---|---|---|
| Year | Country/area | affected | Strain |
| 1959 | Scotland | chicken | H5N1 |
| 1963 | England | turkey | H7N3 |
| 1966 | Ontario (Canada) | turkey | H5N9 |
| 1976 | Victoria (Australia) | chicken | H7N7 |
| 1979 | Germany | chicken | H7N7 |
| 1979 | England | turkey | H7N7 |
| 1983-1985 | Pennsylvania (USA) | chicken, turkey | H5N2 |
| 1983 | Ireland | turkey | H5N8 |
| 1985 | Victoria (Australia) | chicken | H7N7 |
| 1991 | England | turkey | H5N1 |
| 1992 | Victoria (Australia) | chicken | H7N3 |
| 1994 | Queensland (Australia) | chicken | H7N3 |
| 1994-1995 | Mexico | chicken | H5N2 |
| 1994 | Pakistan | chicken | H7N3 |
| 1997 | New South Wales (Australia) | chicken | H7N4 |
| 1997 | Hong Kong (China) | chicken | H5N1 |
| 1997 | Italy | chicken | H5N2 |
| 1999-2000 | Italy | turkey | H7N1 |
| 2002 | Hong Kong (China) | chicken | H5N1 |
| 2002 | Chile | chicken | H7N3 |
| 2003 | Netherlands | chicken | H7N7 |

The skilled person will thus be able to identify future HPAI strains as and when they emerge.

Strains such as A/Duck/Singapore/97 (H5N3) are not HPAI strains.

The avian influenza strain may be of any suitable hemagglutinin subtype, including H1, H2, H3, H4, H5, H6, H7, H8, H9, H10, H11, H12, H13, H14, H15 or H16.

The vaccines of the invention may comprise two or more (i.e., two, three, four, or five) avian influenza strains. Such avian influenza strains may comprise the same or different hemagglutinin subtypes.

The avian virus is not capable of human-to-human transmission.

### The Antigen

Vaccines of the invention include an antigen from a pathogenic avian strain. The antigen will generally be included in a sub-virion form e.g. in the form of a split virus, where the viral lipid envelope has been dissolved or disrupted, or in the form of one or more purified viral proteins. The vaccine composition will contain a sufficient amount of the antigen(s) to produce an immunological response in the patient.

Methods of splitting influenza viruses are well known in the art e.g. see WO02/28422, WO02/067983, WO02/074336, WO01/21151, etc. Splitting of the virus is carried out by disrupting or fragmenting whole virus, whether infectious (wild-type or attenuated) or non-infectious (e.g. inactivated), with a disrupting concentration of a splitting agent. The disruption results in a full or partial solubilisation of the virus proteins, altering the integrity of the virus. Preferred splitting agents are non-ionic and ionic (e.g. cationic) surfactants e.g. alkylglycosides, alkylthioglycosides, acyl sugars, sulphobetaines, betains, polyoxyethylenealkylethers, N,N-dialkyl-Glucamides, Hecameg, alkylphenoxy-polyethoxyethanols, quaternary ammonium compounds, sarcosyl, CTABs (cetyl trimethyl ammonium bromides), tri-N-butyl phosphate, Cetavlon, myristyltrimethylammonium salts, lipofectin, lipofectamine, and DOT-MA, the octyl- or nonylphenoxy polyoxyethanols (e.g. the Triton surfactants, such as Triton X-100 or Triton N101), polyoxyethylene sorbitan esters (the Tween surfactants), polyoxyethylene ethers, polyoxyethlene esters, etc. The BEGRIVAC™, FLUARIX™, FLUZONE™ and FLUESHIELD™ products are split vaccines.

Methods of purifying individual proteins from influenza viruses are well known. Vaccines based on purified viral proteins typically include the hemagglutinin (HA) protein, and often include the neuraminidase (N) protein as well. Processes for preparing these proteins in purified form are well known in the art. The FLUVIRIN™, AGRIPPAL™ and INFLUVAC™ products are subunit vaccines.

As a further alternative, the vaccine may include a whole virus e.g. a live attenuated whole virus or, preferably, an inactivated whole virus. Preferably, the whole virus will not be from the pathogenic avian strain itself, particularly where egg culture is used, but will be a chimeric virus that includes a RNA segment encoding the avian antigen in place of one of its own RNA segments. Vaccines of the invention may thus include a chimeric whole virus, in which at least one of the viral proteins (e.g. the HA) is from a pathogenic avian strain. Methods of inactivating or killing viruses to destroy their ability to infect mammalian cells are known in the art. Such methods include both chemical and physical means. Chemical means for inactivating a virus include treatment with an effective amount of one or more of the following agents: detergents, formaldehyde, formalin, β-propiolactone, or UV light. Additional chemical means for inactivation include treatment with methylene blue, psoralen, carboxyfullerene (C60) or a combination of any thereof. Other methods of viral inactivation are known in the art, such as for example binary ethylamine, acetyl ethyleneimine, or gamma irradiation. The INFLEXAL™ product is a whole cell inactivated vaccine.

In all types of vaccine, dosage is typically normalised to 15 μg of HA per strain per dose, but lower doses may also be used (see below). Normalisation of doses is generally achieved by measuring concentrations using a single radial immunodiffusion (SRID) assay.

Further details on influenza vaccine antigens can be found in chapters 17 & 18 of Vaccines (eds. Plotkin & Orenstein, 4th edition, 2004, ISBN 0-7216-9688-0).

### Viral Growth for Antigen Preparation

Production of vaccines of the invention requires growth of influenza virus, with antigens being prepared from the grown viruses. There are two general methods currently used for influenza virus production: (1) growth of viruses in eggs; (2) growth of viruses in cell culture. Either growth method can be used according to the invention.

Growth on embryonated hen eggs, followed by purification of viruses from allantoic fluid, is the method by which influenza virus has traditionally been grown for vaccine production. More recently, viruses have been grown on cultured cell lines, which avoids the need to prepare virus strains that are adapted to growth on eggs and avoids contamination of the final vaccine with egg proteins. Growth in cell culture is a preferred method for preparing vaccines of the invention. Cells for viral growth may be cultured in suspension or in adherent conditions.

Cell lines suitable for growth of influenza virus are preferably of mammalian origin, and include but are not limited to: human or non-human primate cells (e.g. MRC-5 (ATCC CCL-171), WI-38 (ATCC CCL-75), human embryonic kidney cells (293 cells, typically transformed by sheared adenovirus type 5 DNA), VERO cells from monkey kidneys), horse, cow (e.g. MDBK cells), sheep, dog (e.g. MDCK cells from dog kidneys, ATCC CCL34 MDCK (NBL2) or MDCK 33016, deposit number DSM ACC 2219 as described in WO97/37001), cat, and rodent (e.g. hamster cells such as BHK21-F, HKCC cells, or Chinese hamster ovary cells (CHO cells)), and may be obtained from a wide variety of developmental stages, including for example, adult, neonatal, fetal, and embryo. In certain embodiments the cells are immortalized (e.g. PERC.6 cells, as described in WO01/38362 and WO02/40665, and as deposited under ECACC deposit number 96022940). In preferred embodiments, mammalian cells are utilized, and may be selected from and/or derived from one or more of the following non-limiting cell types: fibroblast cells (e.g. dermal, lung), endothelial cells (e.g. aortic, coronary, pulmonary, vascular, dermal microvascular, umbilical), hepatocytes, keratinocytes, immune cells (e.g. T cell, B cell, macrophage, NK, dendritic), mammary cells (e.g. epithelial), smooth muscle cells (e.g. vascular, aortic, coronary, arterial, uterine, bronchial, cervical, retinal pericytes), melanocytes, neural cells (e.g. astrocytes), prostate cells (e.g. epithelial, smooth muscle), renal cells (e.g. epithelial, mesangial, proximal tubule), skeletal cells (e.g. chondrocyte, osteoclast, osteoblast), muscle cells (e.g. myoblast, skeletal, smooth, bronchial), liver cells, retinoblasts, and stromal cells. WO97/37000 and WO97/37001 describe production of animal cells and cell lines that capable of growth in suspension and in serum free media and are useful in the production and replication of viruses.

Culture conditions for the above cell types are well-described in a variety of publications, or alternatively culture medium, supplements, and conditions may be purchased commercially, such as for example, as described in the catalog and additional literature of Cambrex Bioproducts (East Rutherford, N.J.).

In certain embodiments, the host cells used in the methods described herein are cultured in serum free and/or protein free media. A medium is referred to as a serum-free medium in the context of the present invention in which there are no additives from serum of human or animal origin. Protein-free is understood to mean cultures in which multiplication of the cells occurs with exclusion of proteins, growth factors, other protein additives and non-serum proteins, but can optionally include proteins such as trypsin or other proteases that may be necessary for viral growth. The cells growing in such cultures naturally contain proteins themselves.

Known serum-free media include Iscove's medium, Ultra-CHO medium (BioWhittaker) or EX-CELL (JRH Bioscience). Ordinary serum-containing media include Eagle's Basal Medium (BME) or Minimum Essential Medium (MEM) (Eagle, Science, 130, 432 (1959)) or Dulbecco's Modified Eagle Medium (DMEM or EDM), which are ordinarily used with up to 10% fetal calf serum or similar additives. Optionally, Minimum Essential Medium (MEM) (Eagle, Science, 130, 432 (1959)) or Dulbecco's Modified Eagle Medium (DMEM or EDM) may be used without any serum containing supplement. Protein-free media like PF-CHO (JHR Bioscience), chemically-defined media like ProCHO 4CDM (BioWhittaker) or SMIF 7 (Gibco/BRL Life Technologies) and mitogenic peptides like Primactone, Pepticase or HyPep™ (all from Quest International) or lactalbumin hydrolyzate (Gibco and other manufacturers) are also adequately known in the prior art. The media additives based on plant hydrolyzates have the special advantage that contamination with viruses, mycoplasma or unknown infectious agents can be ruled out.

Cell culture conditions (temperature, cell density, pH value, etc.) are variable over a very wide range owing to the suitability of the cell line employed according to the invention and can be adapted to the requirements of particular influenza strains.

The method for propagating virus in cultured cells generally includes the steps of inoculating the cultured cells with the strain to be cultured, cultivating the infected cells for a desired time period for virus propagation, such as for example as determined by virus titer or antigen expression (e.g. between 24 and 168 hours after inoculation) and collecting the propagated virus. The cultured cells are inoculated with a virus (measured by PFU or TCID₅₀) to cell ratio of 1:500 to 1:1, preferably 1:100 to 1:5, more preferably 1:50 to 1:10. The virus is added to a suspension of the cells or is applied to a monolayer of the cells, and the virus is absorbed on the cells for at least 60 minutes but usually less than 300 minutes, preferably between 90 and 240 minutes at 25° C. to 40° C., preferably 28° C. to 37° C. The infected cell culture (e.g. monolayers) may be removed either by freeze-thawing or by enzymatic action to increase the viral content of the harvested culture supernatants. The harvested fluids are then either inactivated or stored frozen. Cultured cells may be infected at a multiplicity of infection (“m.o.i.”) of about 0.0001 to 10, preferably 0.002 to 5, more preferably to 0.001 to 2. Still more preferably, the cells are infected at a m.o.i of about 0.01. Infected cells may be harvested 30 to 60 hours post infection. Preferably, the cells are harvested 34 to 48 hours post infection. Still more preferably, the cells are harvested 38 to 40 hours post infection. Proteases (typically trypsin) are generally added during cell culture to allow viral release, and the proteases can be added at any suitable stage during the culture.

The virus that is grown, and from which antigens are prepared for use in vaccines of the invention, includes an antigen (e.g. the HA protein) from a pathogenic avian strain but, to allow viral growth in standard systems, will not generally itself be a pathogenic avian strain. Generally, therefore, the growth strain will thus be a reassortant derived from two sources: (1) the pathogenic avian strain and (2) a strain that grows well in a chosen growth system. For example, existing vaccines, particularly those prepared from growth in eggs, are often prepared from reassortant strains derived from (1) the antigenic strain of interest and (2) the A/Puerto Rico/8/34 (H1N1) strain.

Reassortant strains can be prepared randomly, by co-culturing the source viruses, or can be prepared rationally, using “reverse genetics” techniques (e.g. see WO91/03552, U.S. Pat. No. 5,166,057, Neumann & Kawaoka (2001) Virology 287(2):243-50). Reverse genetics involves expressing (a) DNA molecules that encode desired viral RNA molecules e.g. from poll promoters, and (b) DNA molecules that encode viral proteins e.g. from po1II promoters, such that expression of both types of DNA in a cell leads to assembly of a complete intact infectious virion. The DNA preferably provides all of the viral RNA and proteins, but it is also possible to use a helper virus to provide some of the RNA and proteins. Plasmid-based methods using separate plasmids for producing each viral RNA are preferred (WO00/60050, WO01/04333, U.S. Pat. No. 6,649,372), and these methods will also involve the use of plasmids to express all or some (e.g. just the PB1, PB2, PA and NP proteins) of the viral proteins. Ambisense techniques have also been disclosed (WO00/53786) and, rather than use separate plasmids for encoding a given viral RNA and the corresponding viral protein, it is possible to use dual poll and po1II promoters to simultaneously code for the viral RNAs and for expressible mRNAs from a single template (WO01/83794; Hoffmann et al. (2000) Virology 267(2):310-7).

### The Antibody Response

Although vaccines of the invention comprise antigens from pathogenic avian strains, they can invoke antibody responses that are capable of neutralising human transmissible viruses. The ability of pathogenic avian strains to achieve this cross-protectivity was unexpected.

Methods for assessing antibody responses, neutralising capability and protection after influenza virus vaccination are well known in the art. Human studies have shown that antibody titres against hemagglutinin of human influenza virus are correlated with protection (a serum sample hemagglutination-inhibition titre of about 30-40 gives around 50% protection from infection by a homologous virus) {(Potter & Oxford (1979) Br Med Bull 35: 69-75}. Antibody responses are typically measured by hemagglutination inhibition, by microneutralisation, by single radial immunodiffusion (SRI)), and/or by single radial hemolysis (SRH). These assay techniques are well known in the art.

### The Vaccine

Annual human influenza vaccines typically include more than one influenza strain, with trivalent vaccines being normal (e.g. two influenza A virus antigens, and one influenza B virus antigen). In pandemic years, however, a single monovalent strain may be used. Thus the pathogenic avian antigen(s) described above may be the sole influenza antigen(s) in a vaccine of the invention, or the vaccine may additionally comprise antigen(s) from one or more (e.g. 1, 2, 3, 4 or more) further annual influenza virus strains. Specific vaccines of the invention thus include: (i) a vaccine comprising the pathogenic avian antigen(s) as the sole influenza antigen(s); (ii) a vaccine comprising the pathogenic avian antigen(s) plus antigen(s) from two other strains, preferably such that the three strains cover both influenza A and B viruses, and more preferably with two A viruses and one B virus; (iii) a vaccine comprising the pathogenic avian antigen(s) plus antigen(s) from three other strains, wherein said three other strains are two influenza A strains and one influenza B strain.

Traditional human vaccines contain 15 μg of HA per strain per dose, but lower doses have also been shown to be effective (e.g. see WO00/15251, U.S. Pat. No. 6,372,223, WO01/22992, Nicholson et al. (2001) The Lancet 357:1937-1943, Treanor et al. (2002) Vaccine 20:1099-1105), particularly when an adjuvant is used. Thus, vaccines of the invention may comprise between 0.1 μg and 25 μg or 30 μg of HA per strain per dose. The amount of HA for each strain is preferably about the same. Typical μg amounts of each HA for inclusion are about 15, 10, 9, 8, 7.5, 7, 6.5, 6, 5.5, 5, 4.5, 4, 3.5, 3, 2.5, 2, 1.5, 1, 0.5, etc. A preferred set of vaccines comprises an antigen content of between 0.1 and 5 μg HA per strain per dose.

Vaccines of the invention may be formulated for delivery by various routes e.g. by intramuscular injection, by subcutaneous delivery, by intranasal delivery (e.g. WO00/47222, U.S. Pat. No. 6,635,246, WO01/21151, INFLEXAL™, FLUMIST™), by oral delivery (eg. U.S. Pat. No. 6,635,246), by intradermal delivery (e.g. WO02/074336, WO02/067983, WO02/087494, WO02/083214, WO2004/016281), by transdermal delivery, by transcutaneous delivery, by topical routes, etc. Injection may involve a needle (including a microneedle), or may be needle-free. Immunization through certain delivery routes may be enhanced by the use of adjuvants (discussed below).

Vaccines of the invention preferably contain <50 pg/dose of DNA derived from the growth host (e.g. from eggs or from the growth cell line). A convenient method for reducing host cell DNA contamination is disclosed in European patent 0870508 and U.S. Pat. No. 5,948,410, involving a two-step treatment, first using a DNase (e.g. Benzonase) and then a cationic detergent (e.g. CTAB).

Vaccines of the invention may include an antibiotic or other preservative. Preferred vaccines avoid the use of mercurial preservatives, such as thimerosal (also known as merthiolate or thiomersal) and timerfonate. Thus preferred vaccines are substantially free (<5μg/ml) or, more preferably, totally free of mercurial preservative. (Multidose formulations, however, preferably contain an effective amount of preservative).

### Adjuvants

Vaccines of the invention may be administered in conjunction with other immunoregulatory agents. In particular, compositions will usually include an adjuvant. Adjuvants for use with the invention include, but are not limited to, one or more of the following set forth below:

### A. Mineral Containing Compositions

Mineral containing compositions suitable for use as adjuvants in the invention include mineral salts, such as aluminum salts and calcium salts. The invention includes mineral salts such as hydroxides (e.g. oxyhydroxides), phosphates (e.g. hydroxyphosphates, orthophosphates), sulfates, etc. (e.g. see chapters 8 & 9 of Vaccine Design . . . (1995) eds. Powell & Newman. ISBN: 030644867X. Plenum.), or mixtures of different mineral compounds (e.g. a mixture of a phosphate and a hydroxide adjuvant, optionally with an excess of the phosphate), with the compounds taking any suitable form (e.g. gel, crystalline, amorphous, etc.), and with adsorption to the salt(s) being preferred. The mineral containing compositions may also be formulated as a particle of metal salt (WO/0023 105).

Aluminum salts may be included in vaccines of the invention such that the dose of Al³⁺ is between 0.2 and 1.0 mg per dose.

### B. Oil-Emulsions

Oil-emulsion compositions suitable for use as adjuvants in the invention include squalene-water emulsions, such as MF59 (5% Squalene, 0.5% Tween 80, and 0.5% Span 85, formulated into submicron particles using a microfluidizer). See WO90/14837. See also, Podda, “The adjuvanted influenza vaccines with novel adjuvants: experience with the MF59-adjuvanted vaccine”, Vaccine (2001) 19: 2673-2680. MF59 is used as the adjuvant in the FLUAD™ influenza virus trivalent subunit vaccine.

Particularly preferred adjuvants for use in the compositions are submicron oil-in-water emulsions. Preferred submicron oil-in-water emulsions for use herein are squalene/water emulsions optionally containing varying amounts of MTP-PE, such as a submicron oil-in-water emulsion containing 4-5% w/v squalene, 0.25-1.0% w/v Tween 80™ (polyoxyelthylenesorbitan monooleate), and/or 0.25-1.0% Span 85™ (sorbitan trioleate), and, optionally, N-acetylmuramyl-L-alanyl-D-isogluatminyl-L-alanine-2-(1′-2′-dipalmitoyl-sn-glycero-3-huydroxyphosphophoryloxy)-ethylamine (MTP-PE), for example, the submicron oil-in-water emulsion known as “MF59” (International Publication No. WO90/14837; U.S. Pat. Nos. 6,299,884 and 6,451,325, incorporated herein by reference in their entireties; and Ott et al., “MF59—Design and Evaluation of a Safe and Potent Adjuvant for Human Vaccines” in Vaccine Design: The Subunit and Adjuvant Approach (Powell, M. F. and Newman, M. J. eds.) Plenum Press, New York, 1995, pp. 277-296). MF59 contains 4-5% w/v Squalene (e.g. 4.3%), 0.25-0.5% w/v Tween 80™, and 0.5% w/v Span 85™ and optionally contains various amounts of MTP-PE, formulated into submicron particles using a microfluidizer such as Model 110Y microfluidizer (Microfluidics, Newton, MA). For example, MTP-PE may be present in an amount of about 0-500 μg/dose, more preferably 0-250 μg/dose and most preferably, 0-100 μg/dose. As used herein, the term “MF59-0” refers to the above submicron oil-in-water emulsion lacking MTP-PE, while the term MF59-MTP denotes a formulation that contains MTP-PE. For instance, “MF59-100” contains 100 μg MTP-PE per dose, and so on. MF69, another submicron oil-in-water emulsion for use herein, contains 4.3% w/v squalene, 0.25% w/v Tween 80™, and 0.75% w/v Span 85™ and optionally MTP-PE. Yet another submicron oil-in-water emulsion is MF75, also known as SAF, containing 10% squalene, 0.4% Tween 80™, 5% pluronic-blocked polymer L121, and thr-MDP, also microfluidized into a submicron emulsion. MF75-MTP denotes an MF75 formulation that includes MTP, such as from 100-400 μg MTP-PE per dose.

Submicron oil-in-water emulsions, methods of making the same and immunostimulating agents, such as muramyl peptides, for use in the compositions, are described in detail in International Publication No. WO90/14837 and U.S. Pat. Nos. 6,299,884 and 6,45 1,325, incorporated herein by reference in their entireties.

Complete Freund's adjuvant (CFA) and incomplete Freund's adjuvant (IFA) may also be used as adjuvants in the invention.

### C. Saponin Formulations

Saponin formulations, may also be used as adjuvants in the invention. Saponins are a heterologous group of sterol glycosides and triterpenoid glycosides that are found in the bark, leaves, stems, roots and even flowers of a wide range of plant species. Saponin from the bark of the Quillaia saponaria Molina tree have been widely studied as adjuvants. Saponin can also be commercially obtained from Smilax ornata (sarsaprilla), Gypsophilla paniculata (brides veil), and Saponaria officianalis (soap root). Saponin adjuvant formulations include purified formulations, such as QS21, as well as lipid formulations, such as ISCOMs.

Saponin compositions have been purified using High Performance Thin Layer Chromatography (HP-LC) and Reversed Phase High Performance Liquid Chromatography (RP-HPLC). Specific purified fractions using these techniques have been identified, including QS7, QS17, QS18, QS21, QH-A, QH-B and QH-C. Preferably, the saponin is QS21. A method of production of QS21 is disclosed in U.S. Pat. No. 5,057,540. Saponin formulations may also comprise a sterol, such as cholesterol (see WO96/33739).

Combinations of saponins and cholesterols can be used to form unique particles called Immunostimulating Complexs (ISCOMs). ISCOMs typically also include a phospholipid such as phosphatidylethanolamine or phosphatidylcholine. Any known saponin can be used in ISCOMs. Preferably, the ISCOM includes one or more of Quil A, QHA and QHC. ISCOMs are further described in EP0109942, WO96/11711 and WO96/33739. Optionally, the ISCOMS may be devoid of additional detergent. See WO00/07621.

A review of the development of saponin based adjuvants can be found at Barr, et al., “ISCOMs and other saponin based adjuvants”, Advanced Drug Delivery Reviews (1998) 32:247-271. See also Sjolander, et al., “Uptake and adjuvant activity of orally delivered saponin and ISCOM vaccines”, Advanced Drug Delivery Reviews (1998) 32:321-338.

### D. Virosomes and Virus Like Particles (VLPs)

Virosomes and Virus Like Particles (VLPs) can also be used as adjuvants in the invention. These structures generally contain one or more proteins from a virus optionally combined or formulated with a phospholipid. They are generally non-pathogenic, non-replicating and generally do not contain any of the native viral genome. The viral proteins may be recombinantly produced or isolated from whole viruses. These viral proteins suitable for use in virosomes or VLPs include proteins derived from influenza virus (such as HA or NA), Hepatitis B virus (such as core or capsid proteins), Hepatitis E virus, measles virus, Sindbis virus, Rotavirus, Foot-and-Mouth Disease virus, Retrovirus, Norwalk virus, human Papilloma virus, HIV, RNA-phages, QB-phage (such as coat proteins), GA-phage, fr-phage, AP205 phage, and Ty (such as retrotransposon Ty protein pl). VLPs are discussed further in WO03/024480, WO03/024481, and Niikura et al., “Chimeric Recombinant Hepatitis E Virus-Like Particles as an Oral Vaccine Vehicle Presenting Foreign Epitopes”, Virology (2002) 293:273-280; Lenz et al., “Papillomarivurs-Like Particles Induce Acute Activation of Dendritic Cells”, Journal of Immunology (2001) 5246-5355; Pinto, et al., “Cellular Immune Responses to Human Papillomavirus (HPV)-16 Li Healthy Volunteers Immunized with Recombinant HPV-16 LI Virus-Like Particles”, Journal of Infectious Diseases (2003) 188:327-338; and Gerber et al., “Human Papillomavrisu Virus-Like Particles Are Efficient Oral Immunogens when Coadministered with Escherichia coli Heat-Labile Entertoxin Mutant R192G or CpG”, Journal of Virology (2001) 75(10):4752-4760. Virosomes are discussed further in, for example, Gluck et al., “New Technology Platforms in the Development of Vaccines for the Future”, Vaccine (2002) 20:B1O -B16. Immunopotentiating reconstituted influenza virosomes (IRIV) are used as the subunit antigen delivery system in the intranasal trivalent INFLEXAL™ product {Mischler & Metcalfe (2002) Vaccine 20 Suppl 5:B17-23} and the INFLUVAC PLUS™ product.

### E. Bacterial or Microbial Derivatives

Adjuvants suitable for use in the invention include bacterial or microbial derivatives such as:
(1) Non-toxic derivatives of enterobacterial lipopolysaccharide (LPS)

Such derivatives include Monophosphoryl lipid A (MPL) and 3-O-deacylated MPL (3 dMPL). 3 dMPL is a mixture of 3 De-O-acylated monophosphoryl lipid A with 4, 5 or 6 acylated chains. A preferred “small particle” form of 3 De-O-acylated monophosphoryl lipid A is disclosed in EP 0 689 454. Such “small particles” of 3 dMPL are small enough to be sterile filtered through a 0.22 micron membrane (see EP 0 689 454). Other non-toxic LPS derivatives include monophosphoryl lipid A mimics, such as aminoalkyl glucosaminide phosphate derivatives e.g. RC-529. See Johnson et al. (1999) Bioorg Med Chem Lett 9:2273-2278.
(2) Lipid A Derivatives

Lipid A derivatives include derivatives of lipid A from Escherichia coli such as OM-174. OM-174 is described for example in Meraldi et al., “OM-174, a New Adjuvant with a Potential for Human Use, Induces a Protective Response with Administered with the Synthetic C-Terminal Fragment 242-310 from the circumsporozoite protein of Plasmodium berghei”, Vaccine (2003) 21:2485-2491; and Pajak, et al., “The Adjuvant OM-174 induces both the migration and maturation of murine dendritic cells in vivo”, Vaccine (2003) 21:836-842.
(3) Immunostimulatory oligonucleotides

Immunostimulatory oligonucleotides suitable for use as adjuvants in the invention include nucleotide sequences containing a CpG motif (a sequence containing an unmethylated cytosine followed by guanosine and linked by a phosphate bond). Bacterial double stranded RNA or oligonucleotides containing palindromic or poly(dG) sequences have also been shown to be immunostimulatory.

The CpG's can include nucleotide modifications/analogs such as phosphorothioate modifications and can be double-stranded or single-stranded. Optionally, the guanosine may be replaced with an analog such as 2′-deoxy-7-deazaguanosine. See Kandimalla, et al., “Divergent synthetic nucleotide motif recognition pattern: design and development of potent immunomodulatory oligodeoxyribonucleotide agents with distinct cytokine induction profiles”, Nucleic Acids Research (2003) 31(9): 2393-2400; WO02/26757 and WO99/62923 for examples of possible analog substitutions. The adjuvant effect of CpG oligonucleotides is further discussed in Krieg, “CpG motifs: the active ingredient in bacterial extracts?”, Nature Medicine (2003) 9(7): 831-835; McCluskie, et al., “Parenteral and mucosal prime-boost immunization strategies in mice with hepatitis B surface antigen and CpG DNA”, FEMS Immunology and Medical Microbiology (2002) 32:179-185; W098/40100; U.S. Pat. No. 6,207,646; U.S. Pat. No. 6,239,116 and U.S. Pat. No. 6,429,199.

The CpG sequence may be directed to TLR9, such as the motif GTCGTT or TTCGTT. See Kandimalla, et al., “Toll-like receptor 9: modulation of recognition and cytokine induction by novel synthetic CpG DNAs”, Biochemical Society Transactions (2003) 31 (part 3): 654-658. The CpG sequence may be specific for inducing a ThI immune response, such as a CpG-A ODN, or it may be more specific for inducing a B cell response, such a CpG-B ODN. CpG-A and CpG-B ODNs are discussed in Blackwell, et al., “CpG-A-Induced Monocyte IFN-gamma-Inducible Protein-10 Production is Regulated by Plasmacytoid Dendritic Cell Derived IFN-alpha”, J. Immunol. (2003) 170(8):4061-4068; Krieg, “From A to Z on CpG”, TRENDS in Immunology (2002) 23(2): 64-65 and WOO1/95935. Preferably, the CpG is a CpG-A ODN.

Preferably, the CpG oligonucleotide is constructed so that the 5′ end is accessible for receptor recognition. Optionally, two CpG oligonucleotide sequences may be attached at their 3′ ends to form “immunomers”. See, for example, Kandimalla, et al., “Secondary structures in CpG oligonucleotides affect immunostimulatory activity”, BBRC (2003) 306:948-953; Kandimalla, et al., “Toll-like receptor 9: modulation of recognition and cytokine induction by novel synthetic GpG DNAs”, Biochemical Society Transactions (2003) 31(part 3):664-658; Bhagat et al., “CpG penta- and hexadeoxyribonucleotides as potent immunomodulatory agents” BBRC (2003) 300:853-861 and WO03/035836.
(4) ADP-ribosylating toxins and detoxified derivatives thereof.

Bacterial ADP-ribosylating toxins and detoxified derivatives thereof may be used as adjuvants in the invention. Preferably, the protein is derived from E. coli (i.e., E. coli heat labile enterotoxin “LT), cholera (“CT”), or pertussis (“PT”). The use of detoxified ADP-ribosylating toxins as mucosal adjuvants is described in WO95/17211 and as parenteral adjuvants in WO98/42375. Preferably, the adjuvant is a detoxified LT mutant such as LT-K63, LT-R72, and LTR192G. The use of ADP-ribosylating toxins and detoxified derivaties thereof, particularly LT-K63 and LT-R72, as adjuvants can be found in the following references, each of which is specifically incorporated by reference herein in their entirety: Beignon, et al., “The LTR72 Mutant of Heat-Labile Enterotoxin of Escherichia coli Enahnces the Ability of Peptide Antigens to Elicit CD4+T Cells and Secrete Gamma Interferon after Coapplication onto Bare Skin”, Infection and Immunity (2002) 70(6):3012-3019; Pizza, et al., “Mucosal vaccines: non toxic derivatives of LT and CT as mucosal adjuvants”, Vaccine (2001) 19:2534-2541; Pizza, et al., “LTK63 and LTR72, two mucosal adjuvants ready for clinical trials” Int. J. Med. Microbiol (2000) 290(4-5):455- 461; Scharton-Kersten et al., “Transcutaneous Imrnunization with Bacterial ADP-Ribosylating Exotoxins, Subunits and Unrelated Adjuvants”, Infection and Immunity (2000) 68(9):5306-5313; Ryan et al., “Mutants of Escherichia coli Heat-Labile Toxin Act as Effective Mucosal Adjuvants for Nasal Delivery of an Acellular Pertussis Vaccine: Differential Effects of the Nontoxic AB Complex and Enzyme Activity on Th1 and Th2 Cells” Infection and Immunity (1999) 67(12):6270-6280; Partidos et al., “Heat-labile enterotoxin of Escherichia coli and its site-directed mutant LTK63 enhance the proliferative and cytotoxic T-cell responses to intranasally co-immunized synthetic peptides”, Immunol. Lett. (1999) 67(3):209-216; Peppoloni et al., “Mutants of the Escherichia coli heat-labile enterotoxin as safe and strong adjuvants for intranasal delivery of vaccines”, Vaccines (2003) 2(2):285-293; and Pine et al., (2002) “Intranasal immunization with influenza vaccine and a detoxified mutant of heat labile enterotoxin from Escherichia coli (LTK63)” J. Control Release (2002) 85(1-3):263-270. Numerical reference for amino acid substitutions is preferably based on the alignments of the A and B subunits of ADP-ribosylating toxins set forth in Domenighini et al., Mol. Microbiol (1995) 15(6):1165-1167, specifically incorporated herein by reference in its entirety.

### F. Bioadhesives and Mucoadhesives

Bioadhesives and mucoadhesives may also be used as adjuvants in the invention. Suitable bioadhesives include esterified hyaluronic acid microspheres (Singh et al. (2001) J. Cont. Rele. 70:267- 276) or mucoadhesives such as cross-linked derivatives of poly(acrylic acid), polyvinyl alcohol, polyvinyl pyrollidone, polysaccharides and carboxymethylcellulose. Chitosan and derivatives thereof may also be used as adjuvants in the invention. E.g. WO99/27960.

### G. Microparticles

Microparticles may also be used as adjuvants in the invention. Microparticles (i.e. a particle of ˜100 nm to ˜150 μm in diameter, more preferably −200 nm to ˜30 μm in diameter, and most preferably ˜500 nm to ˜10 μm in diameter) formed from materials that are biodegradable and non-toxic (e.g. a poly(α-hydroxy acid), a polyhydroxybutyric acid, a polyorthoester, a polyanhydride, a polycaprolactone, etc.), with poly(lactide-co-glycolide) are preferred, optionally treated to have a negatively-charged surface (e.g. with SDS) or a positively-charged surface (e.g. with a cationic detergent, such as CTAB).

### H. Liposomes

Examples of liposome formulations suitable for use as adjuvants are described in U.S. Pat. No. 6,090,406, U.S. Pat. No. 5,916,588, and EP 0 626 169.

### I. Polyoxyethylene Ether and Polyoxyethylene Ester Formulations

Adjuvants suitable for use in the invention include polyoxyethylene ethers and polyoxyethylene esters. WO99/52549. Such formulations further include polyoxyethylene sorbitan ester surfactants in combination with an octoxynol (WO01/21207) as well as polyoxyethylene alkyl ethers or ester surfactants in combination with at least one additional non-ionic surfactant such as an octoxynol (WO01/21152).

Preferred polyoxyethylene ethers are selected from the following group: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, and polyoxyethylene-23-lauryl ether.

### J. Polyphosphazene (PCPP)

PCPP formulations are described, for example, in Andrianov et al., “Preparation of hydrogel microspheres by coacervation of aqueous polyphophazene solutions”, Biomaterials (1998) 19(1-3):109- 115 and Payne et al., “Protein Release from Polyphosphazene Matrices”, Adv. Drug. Delivery Review (1998) 31(3):185-196.

### K. Muramyl Peptides

Examples of muramyl peptides suitable for use as adjuvants in the invention include N-acetyl-muramyl-L-threonyl-D-isoglutamine (thr-MDP), N-acetyl-normuramyl-1-alanyl-d-isoglutamine (nor-MDP), and N-acetylmuramyl-1-alanyl-d-isoglutaminyl-1-alanine-2-(1′-2′-dipalmitoyl-sn-glycero-3-hydroxyphosphoryloxy)-ethylamine MTP-PE).

### L. Imidazoquinolone Compounds.

Examples of imidazoquinolone compounds suitable for use adjuvants in the invention include Imiquamod and its homologues, described further in Stanley, “Imiquimod and the imidazoquinolones: mechanism of action and therapeutic potential” Clin Exp Dermatol (2002) 27(7):571-577 and Jones, “Resiquimod 3M”, Curr Opin Investig Drugs (2003) 4(2):214-218.

The invention may also comprise combinations of aspects of one or more of the adjuvants identified above. For example, the following adjuvant compositions may be used in the invention:
(1) a saponin and an oil-in-water emulsion (WO99/11241);
(2) a saponin (e.g., QS21)+a non-toxic LPS derivative (e.g. 3 dMPL) (see WO94/00153);
(3) a saponin (e.g., QS21)+a non-toxic LPS derivative (e.g. 3 dMPL)+a cholesterol;
(4) a saponin (e.g. QS21)+3 dMPL+IL-12 (optionally+a sterol) (WO98/57659);
(5) combinations of 3dMPL with, for example, QS21 and/or oil-in-water emulsions (See European patent applications 0835318, 0735898 and 0761231);
(6) SAF, containing 10% Squalane, 0.4% Tween 80, 5% pluronic-block polymer L121, and thr-MDP, either microfluidized into a submicron emulsion or vortexed to generate a larger particle size emulsion.
(7) Ribi™ adjuvant system (RAS), (Ribi Immunochem) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™); and
(8) one or more mineral salts (such as an aluminum salt)+a non-toxic derivative of LPS (such as 3 dPML).

### M. Human Immunomodulators

Human immunomodulators suitable for use as adjuvants in the invention include cytokines, such as interleukins (e.g. IL-1, IL-2, IL-4, IL-5, IL-6, IL-7, IL-12, etc.), interferons (e.g. interferon-γ), macrophage colony stimulating factor, and tumor necrosis factor.

Aluminum salts and MF59 are preferred adjuvants for use with injectable influenza vaccines. Bacterial toxins and bioadhesives are preferred adjuvants for use with mucosally-delivered vaccines, such as nasal vaccines.

### Patients

Vaccines of the invention are typically for use against pandemic influenza virus strains, and so preferred patients for receiving the vaccines are the elderly (e.g. ≧50 years old, preferably ≧65 years), the young (e.g. ≦5 years old), hospitalised patients, healthcare workers, armed service and military personnel, pregnant women, the chronically ill, and people travelling abroad. The vaccines are not suitable solely for these groups, however, and may be used more generally in a population.

Children aged 0-3 years generally receive lower influenza vaccine doses (e.g. ½ dose).

### General

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x means, for example, x±10%.

### MODES FOR CARRYING OUT THE INVENTION

Nicholson et al. (2001) The Lancet 357:1937-1943 showed that a vaccine prepared from the non- pathogenic A/Duck/Singapore/97 (H5N3) avian strain of influenza was able to induce antibody levels for cross-protecting against the antigenically-related pathogenic human strain A/Hong Kong/156/97 (H5N1). The same avian strain is able to cross-protect patients against more distant strains, suggesting that future emerging pandemic human strains will be susceptible to antibodies raised against a previous season's pathogenic avian strains. Preferably, the emerging heterologous human influenza vaccine will be within the same hemagglutinin type (i.e., H5 or H9) as the pathogenic avian influenza strain.

When a human pandemic strain emerges, spreading through the population by human-to-human contact, the strain can be collected and its antigens can be characterised. Rather than wait for this characterisation to take place, however, and then wait further for production of vaccine strains, virus grown, vaccine formulation and vaccine distribution, the method of the invention turns to recent pathogenic avian strains that were spreading through the avian population but failed to spread in the human population. Those pathogenic avian strains are used to prepare vaccine production strains e.g. by reverse genetics to transfer the pathogenic avian strain's HA antigen into a human vaccine production starting strain. The resulting strain is then used for human vaccine production in the normal way, and the vaccine is used to vaccinate a human population at risk from the emerging pandemic strain. The vaccine is able to induce antibodies (in particular, heterotypic antibodies) capable of neutralizing antigenically distinct newly emerging human strains.

It will be understood that the invention has been described by way of example only and modifications may be made while remaining within the scope and spirit of the invention.

1. A vaccine for protecting a human patient against infection by a human influenza virus strain, wherein the vaccine comprises a first antigen from an avian influenza virus strain that can cause highly pathogenic avian influenza.

2. The vaccine of claim 1, wherein said first antigen can invoke an antibody response in the patient that is capable of neutralizing the homologous avian influenza strain and an emerging heterologous human influenza strain.

3. The vaccine of claim 2, wherein the emerging heterologous human influenza strain comprises the same hemagglutinin type.

4. The vaccine of claim 3, wherein the hemagglutinin type is selected from the group consisting of H5 and H9.

5. The vaccine of claim 1, further comprising an adjuvant.

6. The vaccine of claim 1, further comprising a second antigen from one or more human influenza virus strains.

7. (canceled)

8. The vaccine of claim 1, wherein the first antigen is in the form of a purified viral protein, a split virus or a whole virus.

9. A method of preparing a vaccine for protecting a human patient against infection by a human influenza virus strain comprising admixing an antigen from an influenza virus strain that can cause highly pathogenic avian influenza with a pharmaceutically acceptable carrier.

10. The method of claim 9, further comprising admixing the antigen with an adjuvant.

11. The method of claim 9, wherein the antigen is derived from a virus grown in eggs or a virus grown in cell culture.

12. (canceled)

13. A method for protecting a human patient against infection by a human influenza virus strain comprising administering to the patient a vaccine that comprises an antigen from an avian influenza virus strain that can cause highly pathogenic avian influenza.

14. A kit comprising: (a) a first container comprising a first antigen from a pathogenic avian influenza virus strain; and (b) a second container comprising a second antigen from one or more human influenza virus strains.

15. The kit of claim 14, wherein container (a) further comprises an adjuvant.

16. The vaccine of claim 6 wherein the second antigen is in the form of a purified viral protein, a split virus or a whole virus.
